(19) United States
(12) Patent Application Publication
(10) Pub. No.: US 2022/0351840 A1
(43) Pub. Date: Nov. 3, 2022

(54) MITIGATING OPEN SPACE HEALTH RISK FACTORS

(71) Applicant: International Business Machines Corporation, Armonk, NY (US)

(72) Inventors: Michael Andrasak, Red Hook, NY (US); Ashish Deshpande, Hopewell Junction, NY (US); Vijai Kalathur, Wappingers Falls, NY (US); Peter J. Rooney, Poughquag, NY (US); Gary R. Picher, Wappingers Falls, NY (US)

(21) Appl. No.: 17/243,649

(22) Filed: Apr. 29, 2021

(51) Int. Cl. G16H 40/20; G16H 40/67; G16H 50/30; G16H 50/80; G05B 15/02; G16Y 10/60; G16Y 40/20; G01D 21/02

(52) U.S. Cl. CPC G16H 40/20; G16H 40/67; G16H 50/30; G16H 50/80; G05B 15/02; G16Y 10/60; G16Y 40/20; G01D 21/02; G16H 50/20

(57) ABSTRACT

A computer-implemented method for determining and mitigating open space health risk factors comprising processors configured for partitioning an open space area into sections based on Internet-of-Things (IoT) devices being present in each of the one or more sections, determining a risk factor score based on sensor data gathered from the one or more IoT devices, determining one or more events occurred in one or more of the sections based on the sensor data, updating the risk factor score for the one or more sections based on the one or more events, and responsive to updating the risk factor score, generating one or more action items to mitigate the events and reduce the risk factor score. The risk factor score includes a time weight based on the events that reduces the risk factor score over time.

### BACKGROUND

The present invention relates generally to the field of mitigating health risks, and more particularly to determining and mitigating open space health risk factors.

In light of the ongoing global pandemic, and as society is getting used to a new normal of how people maneuver around each other in open spaces, there has to be a better way to identify the risk level associated with a section of a building or open space based on recent past activities that occurred there. For example, in a grocery store or in an office building, a person suffering from a respiratory illness might have coughed or sneezed in certain areas along the route that they traveled, which in turn increases the chance that infectious aerosolized particles are still lingering along that traveled route in that space. Other people who may later, but within a period of time when the aerosolized particles are still present, walk along the same route or within the same area might not be aware of the coughing or sneezing event. Their lack of awareness coupled with their walking through that area increases the chance of them becoming infected by inhaling or ingesting the infectious aerosolized particles.

### SUMMARY

Embodiments described herein provide solutions to mitigate the health risks associated with navigating open spaces where recently occurring events increased the health risk factors in those open spaces. Disclosed are models to identify health risk factors for different areas of a building or open space based on events occurring in those areas. Events may include coughing events, sneezing events, voice projection events, mask events, or temperature events.

Aspects of the present invention disclose a computer-implemented method, computer program product and a computer system for mitigating open space health risk factors. In an embodiment, the computer-implemented method may include one or more processors configured for partitioning an open space area into one or more sections based on one or more Internet-of-Things (IoT) devices being present in each of the one or more sections. Further, the one or more processors may be configured for gathering sensor data from each of the one or more IoT devices corresponding to the one or more sections. Further, the one or more processors may be configured for determining a risk factor score based on the sensor data for each of the one or more sections. Further, the one or more processors may be configured for determining one or more events occurred in one or more of the sections based on the sensor data; updating the risk factor score for the one or more sections based on the one or more events; and responsive to updating the risk factor score, generating one or more action items to mitigate the one or more events and reduce the risk factor score.

In an embodiment, the one or more sections may be defined by boundaries based on a range of the one or more sensors present in the one or more sections. In an embodiment, the one or more sensors may include at least one of a microphone, a temperature sensor, and a camera. The one or more events may include one or more of a cough event, a sneeze event, voice projection event, a mask event, or a temperature event. In an embodiment, the risk factor score may include a time weight based on the one or more events that reduces the risk factor score over time.

In an embodiment, the computer-implemented method for mitigating open space health risks may further include, responsive to determining the one or more events occurred, illuminating the one or more sections where the one or more events occurred with a light indicative of the one or more events and incrementing the risk factor score for the one or more sections by a predetermined value associated with the one or more events. In an embodiment, generating the one or more actions may further include the one or more processors configured for dispatching one or more resources to the one or more sections where the one or more events occurred; and decreasing the risk factor score for the one or more sections by a predetermined value associated with the one or more resources.

### BRIEF DESCRIPTION OF THE DRAWINGS

FIG. 1 depicts a block diagram of a distributed data processing environment for mitigating open space health risk factors, in accordance with an embodiment of the present invention;

FIG. 2 depicts a partitioned space environment for mitigating open space health risk factors, in accordance with an embodiment of the present invention;

FIG. 3 depicts a flowchart of a computer-implemented method for mitigating open space health risk factors, in accordance with an embodiment of the present invention; and

FIG. 4 depicts a block diagram of a computing device of distributed data processing environment, in accordance with an embodiment of the present invention.

### DETAILED DESCRIPTION

In a smart space environment, IoT devices may be programmed to automatically perform activities within the space in lieu of occupants performing the activities.

Given the current climate, finding different ways to keep people safe is critical. To mitigate open space health risk factors, embodiments of the present invention are configured to gather data collected by IoT devices and process the data to determine what actions can be performed or what resources can be dispatched to a location within a controlled environment to mitigate the risks associated with an event that occurred in a previous time frame. Embodiments of the present invention also disclose computer-implemented methods, computer systems, and computer program products to identify health risk factors for different areas of a building based on health risk events (e.g., coughing, sneezing) occurring. Additional factors like detecting temperature changes and sound patterns can be added to further improve the system. A risk factor score for each grid section of a building may be calculated based on the health risk events that happen in that area in a given time window. The risk factor score may then be used to trigger different actions like alerts and extra sanitation effort for that grid area to help reduce the risk of other people in the building becoming exposed to aerosolized particles resulting from the health risk events. These different actions reduce the risk of other people in the building catching a virus that may be associated with the health risk events. Data associated with the detection and reduction of the health risk events can also be used to safely navigate people around a building or an office setting.

In some embodiments, the size of the grid sections does not have to be static and can be customized based on the building in which the model is deployed. For example, if the system is deployed in a supermarket, the supermarket can be sectioned into aisles along with sections for each specialized department (e.g., produce, meat, seafood, bakery). If the risk factor score for a given aisle rises above a predetermined threshold, the system may be configured to perform mitigation actions (e.g., deploy sanitization resources, close off the section, alert occupants) to reduce the risk factor score back to a healthy and acceptable level. Similar mitigation actions can be performed in an office setting, making adjustments to the office setting environment.

Embodiments described herein include computer-implemented methods to identify users within a particular open space environment, wherein each user may provide information or be provided with information to create a user profile. Information used to create a user profile may include demographic data, biometric data, health data, activity data, or other types of data corresponding to the user and/or the associated user device. Once each user profile is established, a user may be enabled to access a service configured to facilitate user navigation within an open space environment governed by embodiments described herein. Artificial intelligence methods may be used to receive all the relevant data, generate output data corresponding to detecting health risk events, notify users navigating the open space environment about the health risk events, and also recommend actions to mitigate the health risks associated with the health risk events. The actions may be performed by the users or by other occupants of the open space environment. However, embodiments described herein may not require any user data input or be associated with a user device. Rather, users may simply access or view a user interface executing a software application directed to the embodiments described herein to receive local health risk data and proceed within the open space environment accordingly. Further, users may simply view indicators in the open space environment to receive health risk information (e.g., light indicators, barriers to close off sections) to mitigate the risk associated with sections of the open space environment.

IoT device data gathered from the IoT devices within the open space environment may also be processed to determine the state or status of the IoT devices and which actions the IoT devices are configured to perform. Action may include providing notifications (e.g., audible, luminous, haptic) to alert users navigating the open space environment of the health risk events or to dispatch resources to reduce the health risks associated with the health risk events. For example, if sensor data indicates a first event occurred associated with a first user device in a first section of the open space environment, then one or more processors may be configured to process the sensor data to generate output data including a notification to a second user device to avoid the first section of the open space environment. The output data may also include a notification to a custodian of the open space environment to perform actions to reduce the health risk associated with the first event.

In an embodiment, an open space environment may be divided into a grid layout where each section of the grid may be associated with one or more IoT devices (e.g., microphone, temperature sensor) configured to detect activity (e.g., sounds, temperature changes) within range of the one or more IoT devices. Data corresponding to detected activity may be processed by a risk factor analysis model to determine a risk factor score for each section of the grid and trigger follow up actions.

In an embodiment, data (e.g., sensor data) corresponding to the open space environment may be gathered by the IoT devices active in the sections of the open space environment. The gathered data may be processed by the risk factor analysis model. The gathered data may include sound data (e.g., coughs, sneezes, vocal projections) corresponding to excessive release of aerosolized particles may be processed for each section of the open space environment. For each sound corresponding to an elevated health risk detected, the one or more processors may be configured to generate an alert to notify other users within, or custodians of, the open space environment about the health risk event that has occurred including the section of which the health risk event occurred. Further, the gathered data may include temperature data (e.g., change in ambient temperature, elevated user body temperature) corresponding to a user that may be suffering from a fever related to an infectious health condition.

In an embodiment, the one or more processors may be configured to determine (e.g., calculate) a risk factor score based on the sensor data. For example, when an event (e.g., health risk event) is detected, the sensor data is transmitted to the risk factor analysis model and processed to update (e.g., increment by a predetermined amount) the risk factor score for the section where the event was detected. For example, if the event that was detected in a first section is a cough event, then the risk factor score for the first section may be incremented by a predefined value (e.g., 5 for a cough event, 7 for a sneeze event).

In an embodiment, the one or more processors may be configured to apply a time weight to the risk factor score, wherein the time weight may be configured to diminish the risk factor score over time. For example, if the risk factor score for a first section of the open space environment is a total of 5 (e.g., 5 for a cough event), and a time weight for the cough event is a reduction of 2 for the first 15 minutes and 3 for the second 15 minutes, then after 30 minutes has expired, the risk factor score for the first section would be 0. Thus, the time weight for the cough event reduces the risk factor score to 0 after 30 minutes as the potency of the aerosolized particles diminishes over time.

In an embodiment, the one or more processors may be configured to detect a sanitizing event as one of the actions to be performed to mitigate the event associated with an elevated health risk. Further, the one or more processors may be configured to generate an action item corresponding to dispatching a sanitizing service to the section of the open space environment where the elevated health risk event was detected. Once dispatched, the one or more processors may be configured to reduce the risk factor score by a predetermined amount in response to determining that the sanitizing event has occurred. For example, if a cough event was detected in a first section of the open space environment that resulted in a risk factor score of 5, and the one or more processors has detected that a sanitizing event has occurred in the first section, then the one or more processors may be configured to reduce the risk factor score by a predetermined amount (e.g., 4) for the first section. Therefore, the first section risk factor score would be updated to 1 immediately after the sanitizing event has occurred and would be reduced to 0 after 15 minutes due to the time weight reduction of 2 after 15 minutes.

In an embodiment, the one or more processors may be configured to perform one or more action items to mitigate the events associated with an elevated health risk to reduce the risk factor score. In other words, actions can be performed if the risk factor scores for sections of the open space environment reach certain thresholds. For example, the one or more processors may be configured to illuminate the grid section with a colored light (e.g., green=no risk factors, yellow=moderate risk factors, red=severe risk factors). Further, the one or more processors may be configured to restrict access to the section where the risk factor score has elevated to unsafe levels. Further, the one or more processors may be configured to schedule and dispatch additional cleaning or sanitizing for the section where the risk factor score has elevated to unsafe levels. Data corresponding to the detection and mitigation of elevated health risk factors may be processed by the one or more processors to provide notifications and guidance to users and occupants in the open space environment to facilitate safer navigation of the open space environment.

The present invention will now be described in detail with reference to the Figures.

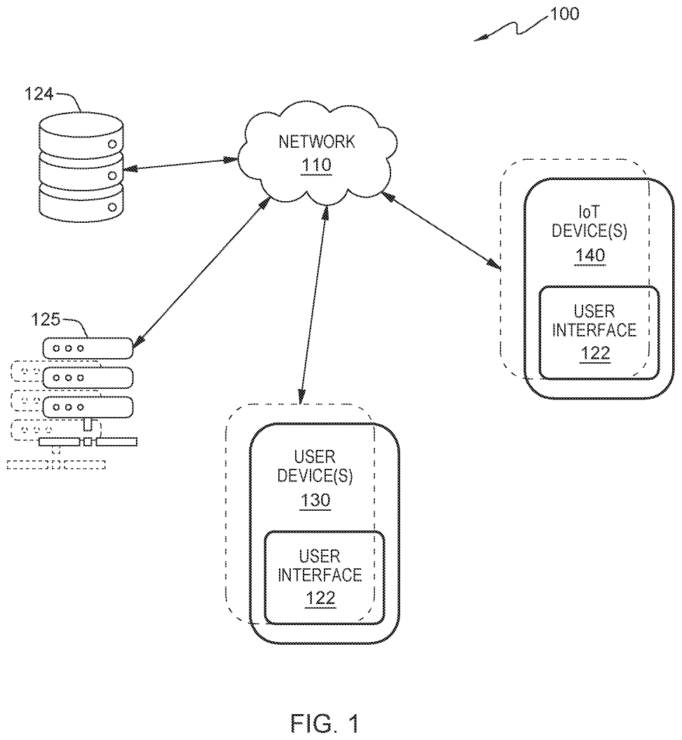

FIG. 1 depicts a block diagram of a distributed data processing environment 100 for mitigating open space health risk factors, in accordance with an embodiment of the present invention.

FIG. 1 provides only an illustration of one embodiment of the present invention and does not imply any limitations with regard to the environments in which different embodiments may be implemented. As shown in FIG. 1, the distributed data processing environment 100 for mitigating activity deficiency includes network 110 configured to facilitate communication between database 124, server 125, user device(s) 130 and IoT device(s) 140. In an example embodiment, one or more processors may be configured for receiving user profile data, device profile data, and sensor data via network 110. Further, one or more processors may be configured for sending output data to user device(s) 130 and IoT device(s) 140 via network 110.

Network 110 operates as a computing network that can be, for example, a local area network (LAN), a wide area network (WAN), or a combination of the two, and can include wired, wireless, or fiber optic connections. In general, network 110 can be any combination of connections and protocols that will support communications between user device(s) 130 and IoT device(s) 140. It is further understood that in some embodiments network 110 is optional and the distributed data processing environment 100 for mitigating open space health risk factors can operate as a stand-alone system, where in other embodiments, network 110 may be configured to enable user device(s) 130 and/or IoT device(s) 140 to share a joint database using network 110.

User interface 122 operates as a local user interface on user device(s) 130 through which one or more users of user device(s) 130 interact with user device(s) 130. User interface 122 may also operate as a local user interface on IoT device(s) 140 through which one or more users of user device(s) 130 interact with IoT device(s) 140. In some embodiments, user interface 122 is a local app interface of a program (e.g., software configured to execute the steps of the invention described herein) on user device(s) 130 or IoT device(s) 140. In some embodiments, user interface 122 is a graphical user interface (GUI), a web user interface (WUI), and/or a voice user interface (VUI) that can display (i.e., visually), present (i.e., audibly), and/or enable a user to enter or receive information (i.e., graphics, text, and/or sound) for or from the program via network 110. In an embodiment, user interface 122 enables a user to send and receive data (i.e., to and from the program via network 110, respectively). In an embodiment, user interface 122 enables a user to opt-in to the program, input user related data, and receive alerts to complete a task or activity.

Database 124 may operate as a repository for data associated with server 125, user device(s) 130, IoT device(s) 140, and other data transmitted within network 110. A database is an organized collection of data. For example, user profile data may include data associated with user device(s) 130. Further, device profile data may include data associated with IoT device(s) 140. Database 124 can be implemented with any type of storage device capable of storing data and configuration files that can be accessed and utilized by either of user device(s) 130 or IoT device(s) 140, such as a database server, a hard disk drive, or a flash memory. In an embodiment, database 124 may be accessed by user device(s) 130 or IoT device(s) 140 to store data associated with user device(s) 130 or IoT device(s) 140. In another embodiment, database 124 may be accessed by user device(s) 130 or IoT device(s) 140 to access data as described herein. In an embodiment, database 124 may reside independent of network 110. In another embodiment, database 124 may reside elsewhere within distributed data processing environment 100 provided database 124 has access to network 110.

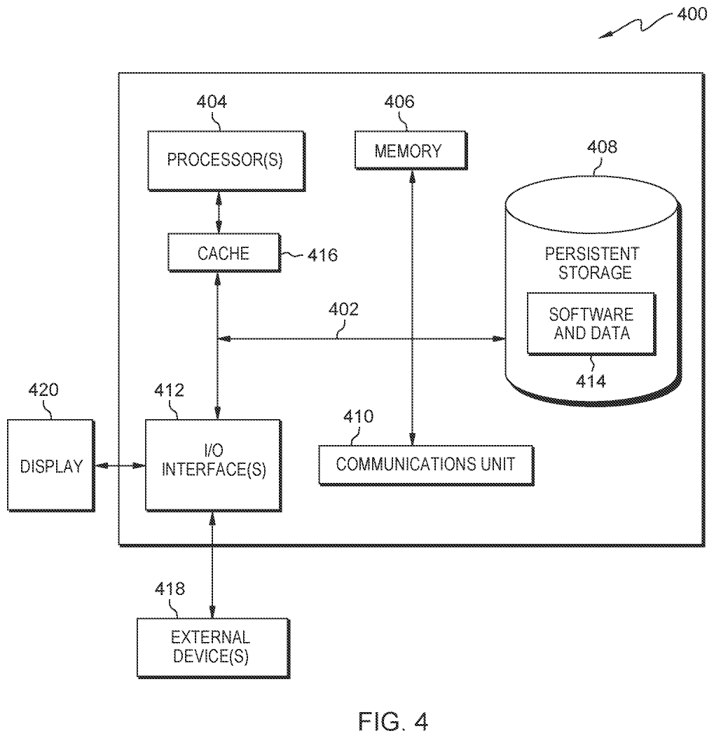

In the depicted embodiment, server(s) 125 may contain a program (e.g., software configured to execute the steps of the invention described herein) and database 124. In some embodiments, server(s) 125 can be a standalone computing device(s), a management server(s), a web server(s), a mobile computing device(s), or any other electronic device(s) or computing system(s) capable of receiving, sending, and processing data. In some embodiments, server 125 may be a laptop computer, tablet computer, netbook computer, personal computer (PC), a desktop computer, a smart phone, or any programmable electronic device capable of communicating with user device(s) 130 and IoT device(s) 140 via network 110. In other embodiments, server(s) 125 represents a server computing system utilizing multiple computers as a server system, such as a cloud computing environment. In yet other embodiments, server(s) 125 represents a computing system utilizing clustered computers and components (e.g., database server computers, application server computers, etc.) that act as a single pool of seamless resources when accessed within distributed data processing environment 100. Server(s) 125 may include components as described in further detail in FIG. 4.

User device(s) 130 may be an electronic device configured for accompaniment with a user. User device(s) 130 may be a personal electronic device such as a mobile communications device, smart phone, tablet, personal digital assistant, smart wearable device, personal laptop computer, desktop computer, or any other electronic device configured for user interaction and gathering user information to generate a user profile. User device(s) 130 may include components as described in further detail in FIG. 4.

IoT device(s) 140 may be an electronic device configured to be a component within a smart environment automation system including lighting systems, heating and air conditioning systems, media, and security systems. The electronic device may include a wireless sensor, software, actuators, and computer devices. IoT device(s) 140 may be embedded in mobile devices, industrial equipment, environmental sensors, medical devices, and others. IoT device(s) 140 may be controlled from a remotely controlled system via network 110 or locally controlled system via a local network, or a combination of both. Further, IoT device(s) 140 may be configured to be controlled via a software application installed and executed by IoT device(s) 140. IoT device(s) 140, when connected to a network, may convey usage data and other types of data corresponding to the device itself, or other devices connected via network 110, wherein the data may provide insights that are useful within the scope of the designed application. IoT device(s) 140 may be configured with a processor, memory, and peripherals (not shown) to receive and process data.

For user device(s) 130, a device profile includes, but is not limited to, a user device identifier (ID), a device type (e.g., a smart watch, a smart phone), data usage patterns for user device(s) 130, and data usage models for user device(s) 130. Data usage patterns may include data type, data use frequency, and user device data use history. A device profile may be created for each user device 130 in network 110. User device(s) 130 may consider data usage patterns and data usage models in a device profile when determining whether to execute a data usage request by user device(s) 130.

For IoT device(s) 140, a device profile includes, but is not limited to, an IoT device identifier (ID), and an IoT device type (e.g., a camera, thermometer). A device profile may be created for each IoT device(s) 140 in network 110.

User device(s) 130 and/or IoT device(s) 140 may operate as physical devices and/or everyday objects that are embedded with electronics, Internet connectivity, and other forms of hardware (e.g., sensors). In general, IoT device(s) 140 can communicate and interact with other IoT device(s) 140 over the Internet, or a local network while being remotely monitored and controlled. Types of IoT device(s) 140 include, but are not limited to, smart cameras, smart thermometers, smart locks, garage doors, refrigerators, freezers, ovens, mobile devices, smart watches, air conditioning (A/C) units, washer/dryer units, smart TVs, virtual assistance devices, and any other smart environment devices.

In an embodiment, a user may be permitted to opt-in and/or agree to a terms and service agreement upon setting up IoT devices with network 110. The terms and service agreement may document the purpose of the information and data sharing between user device(s) 130 or IoT device(s) 140 and provide access to IoT devices on the network that have been designated for participation in network 110. The user agreement may include all mentioned passing devices that would allow control(s), trigger(s), or action(s) to be executed based on the user's original request. For networks with multiple users and multiple IoT devices, the system may extend the usage agreement to a defined or dynamic group, upon a new user joining said group.

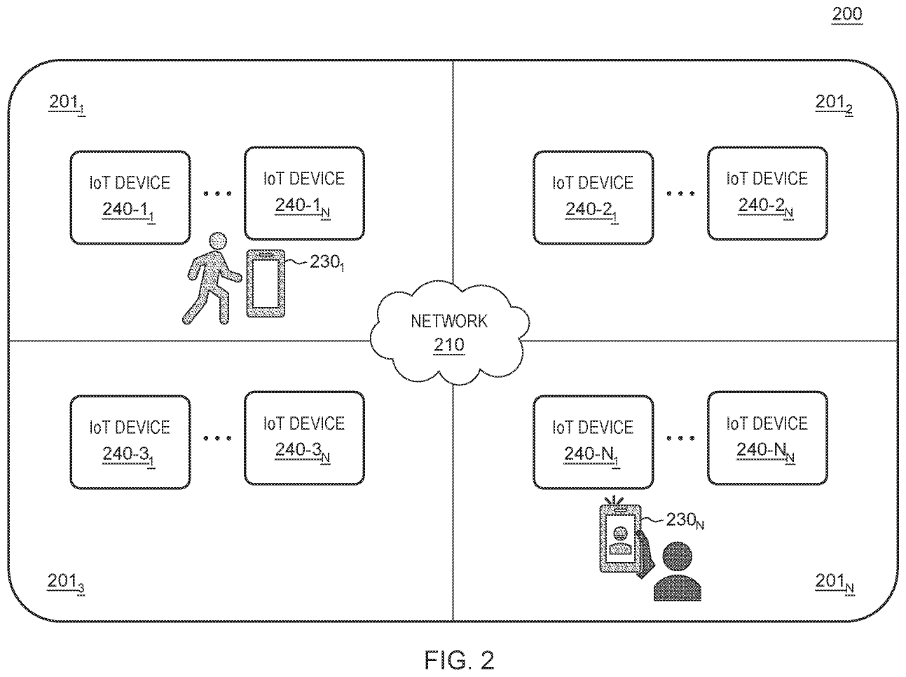

FIG. 2 depicts a partitioned space environment 200 for mitigating activity deficiency, in accordance with an embodiment of the present invention.

In an embodiment, environment 200 may include sections (e.g., 201_{1-N}) each having one or more IoT device(s) (e.g., 240-1_{1-N}, 240-2_{1-N}, 240-3_{1-N} . . . 240-N_{1-N}) in communication with network 210 configured for facilitating communication between one or more user devices (e.g., user device 230_{1-N}) and one or more IoT devices (e.g., 240-1_{1-N}-240-N_{1-N}). User device 230₁ and user device 230_{N} may be configured to operate similar to how user device(s) 130 are described to operate in FIG. 1. IoT devices 240-11-_{N} —240-N_{1-N} may be configured to operate similar to how IoT device(s) 140 are described to operate in FIG. 1. Partitioned space environment 200 may further include database (not shown) configured for storing data transmitted via network 210 and server (not shown) configured for processing data transmitted via network 210. For example, IoT devices (e.g., 240-1 _{1-N}-240-N_{1-N}) of a given open space environment 100 may be placed throughout and connected to network 210 (e.g., Wi-Fi network). IoT devices (e.g., 240-1_{1-N} -240-N_{1-N}) connected to network 210 may include local communication ports that may be in an open state to accept text data. The text data may be processed as text input to a logic engine. The connection to the Wi-Fi network may setup similarly to how a user may setup a smart plug or smart lightbulb, as known to a person of ordinary skill in the art.

In an embodiment, one or more processors may be configured to partition the environment 200 into sections (e.g., 201_{1-N}) defined by a range of one or more IoT device(s) (e.g., 240-1_{1-N}-240-N_{1-N}) present within each section. For example, section 201₁ may include one or more IoT device(s) 240-1_{1-N} configured to detect events that occur within section 201₁. The one or more IoT device(s) (e.g., 240-1_{1-N}-240-N_{1-N}) may include one or more sensors configured to detect events about the surrounding environment within range of the sensors. The one or more IoT devices may include at least one of a microphone, a temperature sensor, a sanitizer, a smart light bulb, and a camera. For example, the sensors may include a camera configured to capture still or moving images. Further, the sensors may include a thermometer configured to detect the temperature of the environment or the temperature of a person within proximity of the thermometer's range. Further, the sensors may include a microphone configured to capture sounds within range of the microphone.

In an embodiment, the one or more sections (e.g., 201_{1-N}) are defined by boundaries based on a range of the one or more IoT devices (e.g., 240-1_{1-N}-240-N_{1-N}) present in the one or more sections (e.g., 201_{1-N}).

In an embodiment, the one or more processors may be configured to gather sensor data from the one or more IoT devices corresponding to the one or more sections. For example, IoT device(s) 240-1_{1-N} may be a microphone configured to capture sensor data corresponding to a cough event detected within a first section 201₁ of environment 200. Further, IoT device(s) 240-1_{1-N} may be a microphone configured to capture sensor data corresponding to a sneeze event detected within a first section 201₁ of environment 200. Further, IoT device(s) 240-1_{1-N} may be a camera configured to capture sensor data corresponding to a mask event detected within a first section 201₁ of environment 200, wherein the mask event is determined by capturing images corresponding to a user removing a mask from their face. Further, IoT device(s) 240-1_{1-N} may be a microphone configured to capture sensor data corresponding to a voice projection event detected within a first section 201₁ of environment 200, wherein the voice projection event is determined by detecting an elevated voice sound (e.g., yelling, screaming, shouting, singing loudly) produced by a user.

In an embodiment, the one or more processors may be configured to determine a risk factor score based on the sensor data for each of the one or more sections 201_{1-N}. For example, if IoT device(s) 240-1_{1-N} in section 201₁ detects no events corresponding to an elevated health risk (e.g., coughing, sneezing, voice projections), then the risk factor score will be 0, indicating no health risk is present at that time in section 201₁.

In an embodiment, the one or more processors may be configured to determine one or more events occurred in one or more sections 201_{1-N} based on the sensor data. The one or more events may include one or more of a cough event, a sneeze event, voice projection event, a mask event, or a temperature event. For example, if the one or more processors process the sensor data and determined that the sensor data corresponds to a coughing sound detected in section 201₁, then the one or more processors will determine that a cough event has occurred in section 201₁.

In an embodiment, the one or more processors may be configured to update the risk factor score for the one or more sections 201_{1-N} based on the one or more events. For example, if the risk factor score for section 201₁ is 0 and a cough event with a risk factor score of 5 is detected in section 201₁, then the risk factor score for section 201₁ is incremented by 5 to generate an updated risk factor score of 5. If additional events are detected in section 201₁, then the risk factor scores associated with the additional events are added to the risk factor score for section 201₁ accordingly.

In an embodiment, responsive to updating the risk factor score, the one or more processors may be configured to generate one or more action items to mitigate the one or more events and reduce the risk factor score. For example, if a cough event is detected in section 201₁ that increases the risk factor score to 5, then the one or more processors may be configured to generate an action item corresponding to dispatching one or more resources to section 201₁ that will decrease the risk factor score. An example of a resource may include sanitizing section 201₁, closing off section 201₁ from other users navigating environment 200, or performing some other activity that will reduce the risk associated with the cough event.

In an embodiment, responsive to determining the one or more events occurred, the one or more processors may be configured to illuminate the one or more sections 201_{1-N} where the one or more events occurred with the smart bulb indicative of the one or more events. For example, if the IoT device(s) 240-1_{1-N} in section 201₁ is a microphone and detects a cough event, the one or more processors may be configured to illuminate section 201₁ with one of the IoT device(s) 240-1_{1-N} that is a smart bulb with a light color corresponding to a health risk factor corresponding to a cough event. The smart bulb may be configured to produce a different color for the different levels of the risk factor score. For example, for a risk factor score of 0, the smart bulb may illuminate a green color. For a risk factor score of 1-5, the smart bulb may illuminate a yellow color. For a risk factor score of 6-10, the smart bulb may illuminate a red color. Further, as additional events occur, the risk factor score may be automatically updated.

In an embodiment, the one or more processors may be configured to apply a time weight to the risk factor score, wherein the time weight may be configured to diminish the risk factor score over time. For example, if the risk factor score for a first section of the open space environment is a total of 5 (e.g., 5 for a cough event), and a time weight for the cough event is a reduction of 2 for the first 15 minutes and 3 for the second 15 minutes, then after 30 minutes has expired, the risk factor score for the first section would be 0. Thus, the time weight for the cough event reduces the risk factor score to 0 after 30 minutes as the potency of the aerosolized particles diminishes over time.

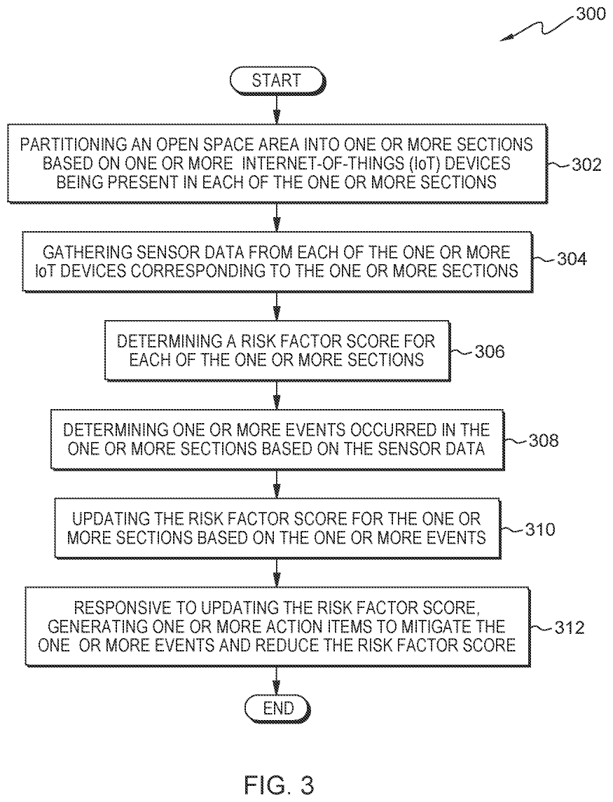

FIG. 3 depicts a flowchart of a computer-implemented method for mitigating open space health risk factors, in accordance with an embodiment of the present invention.

In an embodiment, computer-implemented method 300 may include one or more processors configured for partitioning 302 an open space area into one or more sections based on one or more Internet-of-Things (IoT) devices being present in each of the one or more sections. The one or more sections may be defined by boundaries based on a range of the one or more IoT devices present in the one or more sections.

In an embodiment, computer-implemented method 300 may include one or more processors configured for gathering 304 sensor data from each of the one or more IoT devices corresponding to the one or more sections. The one or more IoT devices include at least one of a microphone, a temperature sensor, a sanitizer, a smart light bulb, and a camera. Other IoT devices configured to detect human activity within an open space environment may be included.

In an embodiment, computer-implemented method 300 may include one or more processors configured for determining 306 a risk factor score for each of the one or more sections. The risk factor score may include a time weight based on the one or more events that reduces the risk factor score over time.

In an embodiment, computer-implemented method 300 may include one or more processors configured for determining 308 one or more events occurred in the one or more sections based on the sensor data. The one or more events may include one or more of a cough event, a sneeze event, voice projection event, a mask event, or a temperature event. Responsive to determining 308 the one or more events occurred, the one or more processors may be configured for illuminating the one or more sections where the one or more events occurred with the smart light bulb indicative of the one or more events. Further, the one or more processors may be configured for incrementing the risk factor score for the one or more sections by a predetermined value associated with the one or more events.

In an embodiment, computer-implemented method 300 may include one or more processors configured for updating 310 the risk factor score for the one or more sections based on the one or more events.

In an embodiment, responsive to updating the risk factor score, computer-implemented method 300 may include one or more processors configured for generating 312 one or more action items to mitigate the one or more events and reduce the risk factor score. Further, the one or more processors may be configured for dispatching one or more resources to the one or more sections where the one or more events occurred and decreasing the risk factor score for the one or more sections by a predetermined value associated with the one or more resources.

FIG. 4 depicts a block diagram of devices 130, 140 of distributed data processing environment, in accordance with an embodiment of the present invention, in accordance with an illustrative embodiment of the present invention. It should be appreciated that FIG. 4 provides only an illustration of one implementation and does not imply any limitations with regard to the environments in which different embodiments may be implemented. Many modifications to the depicted environment may be made.

Devices 400 (e.g., user device(s) 130, IoT device(s) 140) includes communications fabric 402, which provides communications between cache 416, memory 406, persistent storage 408, communications unit 410, and input/output (I/O) interface(s) 412. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses or a crossbar switch.

Memory 406 and persistent storage 408 are computer readable storage media. In this embodiment, memory 406 includes random access memory (RAM). In general, memory 406 can include any suitable volatile or non-volatile computer readable storage media. Cache 416 is a fast memory that enhances the performance of computer processor(s) 404 by holding recently accessed data, and data near accessed data, from memory 406.

Software and data 414 may be stored in persistent storage 408 and in memory 406 for execution and/or access by one or more of the respective computer processors 404 via cache 416. In an embodiment, persistent storage 408 includes a magnetic hard disk drive. Alternatively, or in addition to a magnetic hard disk drive, persistent storage 408 can include a solid-state hard drive, a semiconductor storage device, read-only memory (ROM), erasable programmable read-only memory (EPROM), flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408.

Communications unit 410, in these examples, provides for communications with other data processing systems or devices. In these examples, communications unit 410 includes one or more network interface cards. Communications unit 410 may provide communications through the use of either or both physical and wireless communications links. Software and data 414 may be downloaded to persistent storage 408 through communications unit 410.

I/O interface(s) 412 allows for input and output of data with other devices that may be connected to server 125, user device(s) 130, and/or IoT device(s) 140. For example, I/O interface 412 may provide a connection to external devices 418 such as a keyboard, keypad, a touch screen, and/or some other suitable input device. External devices 418 can also include portable computer readable storage media such as, for example, thumb drives, portable optical or magnetic disks, and memory cards. Software and data 414 used to practice embodiments of the present invention can be stored on such portable computer readable storage media and can be loaded onto persistent storage 408 via I/O interface(s) 412. I/O interface(s) 412 also connect to a display 420.

Display 420 provides a mechanism to display data to a user and may be, for example, a computer monitor.

The present invention may contain various accessible data sources, such as database 124, that may include personal data, content, or information the user wishes not to be processed. Personal data includes personally identifying information or sensitive personal information as well as user information, such as tracking or geolocation information. Processing refers to any, automated or unautomated, operation or set of operations such as collection, recording, organization, structuring, storage, adaptation, alteration, retrieval, consultation, use, disclosure by transmission, dissemination, or otherwise making available, combination, restriction, erasure, or destruction performed on personal data. Software and data 414 may enable the authorized and secure processing of personal data. Software and data 414 may be configured to provide informed consent, with notice of the collection of personal data, allowing the user to opt in or opt out of processing personal data. Consent can take several forms. Opt-in consent can impose on the user to take an affirmative action before personal data is processed. Alternatively, opt-out consent can impose on the user to take an affirmative action to prevent the processing of personal data before personal data is processed. Software and data 414 may provide information regarding personal data and the nature (e.g., type, scope, purpose, duration, etc.) of the processing. Software and data 414 provide the user with copies of stored personal data. Software and data 414 allow the correction or completion of incorrect or incomplete personal data. Software and data 414 allow the immediate deletion of personal data.

The present invention may be a system, a computer-implemented method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of computer-implemented methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method for determining and mitigating open space health risk factors, the computer-implemented method comprising:
partitioning, by one or more processors, an open space area into one or more sections based on one or more Internet-of-Things (IoT) devices being present in each of the one or more sections;
determining, by the one or more processors, a risk factor score based on sensor data gathered from the one or more IoT devices;
determining, by the one or more processors, one or more events occurred in the one or more of the sections based on the sensor data;
updating, by the one or more processors, the risk factor score for the one or more sections based on the one or more events; and
responsive to updating the risk factor score, generating, by the one or more processors, one or more action items to mitigate the one or more events and reduce the risk factor score.

2. The computer-implemented method of claim 1, wherein the one or more sections are defined by boundaries based on a range of the one or more IoT devices present in the one or more sections.

3. The computer-implemented method of claim 1, wherein the one or more IoT devices include at least one of a microphone, a temperature sensor, a sanitizer, a smart light bulb, and a camera.

4. The computer-implemented method of claim 1, wherein the one or more events may include one or more of a cough event, a sneeze event, voice projection event, a mask event, or a temperature event.

5. The computer-implemented method of claim 1, wherein the risk factor score includes a time weight based on the one or more events that reduces the risk factor score over time.

6. The computer-implemented method of claim 3, further comprising:
responsive to determining the one or more events occurred, illuminating, by the one or more processors, the one or more sections where the one or more events occurred with the smart light bulb indicative of the one or more events; and
incrementing the risk factor score for the one or more sections by a predetermined value associated with the one or more events.

7. The computer-implemented method of claim 1, wherein generating the one or more actions further comprises:
dispatching, by the one or more processors, one or more resources to the one or more sections where the one or more events occurred; and
decreasing, by the one or more processors, the risk factor score for the one or more sections by a predetermined value associated with the one or more resources.

8. A computer program product for determining and mitigating open space health risk factors, the computer program product comprising:
one or more computer readable storage media and program instructions stored on the one or more computer readable storage media, the program instructions comprising:
program instructions to partition an open space area into one or more sections based on one or more IoT devices being present in each of the one or more sections;
program instructions to determine a risk factor score based on sensor data gathered from the one or more IoT devices;
program instructions to determine one or more events occurred in the one or more of the sections based on the sensor data;
program instructions to update the risk factor score for the one or more sections based on the one or more events; and
responsive to the program instructions to update the risk factor score, program instructions to generate one or more action items to mitigate the one or more events and reduce the risk factor score.

9. The computer program product of claim 8, wherein the one or more sections are defined by boundaries based on a range of the one or more IoT devices present in the one or more sections.

10. The computer program product of claim 8, wherein the one or more IoT devices include at least one of a microphone, a temperature sensor, a sanitizer, a smart light bulb, and a camera.

11. The computer program product of claim 8, wherein the one or more events may include one or more of a cough event, a sneeze event, voice projection event, a mask event, or a temperature event.

12. The computer program product of claim 8, wherein the risk factor score includes a time weight based on the one or more events that reduces the risk factor score over time.

13. The computer program product of claim 10, further comprising:
responsive to the program instructions to determine the one or more events occurred, program instructions to illuminate the one or more sections where the one or more events occurred with the smart light bulb indicative of the one or more events; and
program instructions to increment the risk factor score for the one or more sections by a predetermined value associated with the one or more events.

14. The computer program product of claim 8, wherein the program instructions to generate the one or more actions further comprises:
program instructions to dispatch one or more resources to the one or more sections where the one or more events occurred; and
program instructions to decrease the risk factor score for the one or more sections by a predetermined value associated with the one or more resources.

15. A computer system for determining and mitigating open space health risk factors, the computer system comprising:
one or more computer processors;
one or more computer readable storage media;
program instructions stored on the one or more computer readable storage media for execution by at least one of the one or more processors, the program instructions comprising:
program instructions to partition an open space area into one or more sections based on one or more IoT devices being present in each of the one or more sections;
program instructions to determine a risk factor score based on sensor data gathered from the one or more IoT devices;
program instructions to determine one or more events occurred in the one or more of the sections based on the sensor data;
program instructions to update the risk factor score for the one or more sections based on the one or more events; and
responsive to the program instructions to update the risk factor score, program instructions to generate one or more action items to mitigate the one or more events and reduce the risk factor score.

16. The computer system of claim 15, wherein the one or more sections are defined by boundaries based on a range of the one or more IoT devices present in the one or more sections.

17. The computer system of claim 15, wherein the one or more sensors include at least one of a microphone, a temperature sensor, a sanitizer, a smart light bulb, and a camera.

18. The computer system of claim 15, wherein the one or more events may include one or more of a cough event, a sneeze event, voice projection event, a mask event, or a temperature event, wherein the risk factor score includes a time weight based on the one or more events that reduces the risk factor score over time.

19. The computer system of claim 17, further comprising:
responsive to the program instructions to determine the one or more events occurred, program instructions to illuminate the one or more sections where the one or more events occurred with the smart light bulb indicative of the one or more events; and
program instructions to increment the risk factor score for the one or more sections by a predetermined value associated with the one or more events.

20. The computer system of claim 15, wherein the program instructions to generate the one or more actions further comprises:
program instructions to dispatch one or more resources to the one or more sections where the one or more events occurred; and
program instructions to decrease the risk factor score for the one or more sections by a predetermined value associated with the one or more resources.
